(19) United States
(12) Patent Application Publication
(10) Pub. No.: US 2008/0299200 A1
(43) Pub. Date: Dec. 4, 2008

(54) Oil-in-Water Emulsion for Creating New Product Consistencies

(75) Inventors: Martin Leser, Bretigny (CH); Laurent Sagalowicz, Cully (CH); Martin Michel, Lausanne (CH); Samuel Guillot, Olivet (FR); Otto Glatter, Graz (AT); Matija Tomsic, Skofja Loka (SI)

Correspondence Address: BELL, BOYD & LLOYD LLP, P.O. Box 1135, CHICAGO, IL, 60690, US

(73) Assignee: NESTEC S.A., Vevey (CH)

(21) Appl. No.: 12/094,690

(22) PCT Filed: Nov. 22, 2006
(86) PCT No.: PCT/EP2006/068753

§ 371 Date: May 22, 2008

(30) Foreign Application Priority Data
Nov. 23, 2005 (EP) 05025526.4

(51) Int. Cl. A61K 9/107; B01J 13/00; C12N 9/00; A23D 7/00

(52) U.S. Cl. 424/484; 516/53; 516/72; 516/68; 435/183; 426/602; 426/534; 516/98

(57) ABSTRACT

The invention concerns an oil-in-water emulsion wherein the oil droplets of a diameter in the range of 5 nm to hundreds of micrometers exhibit a nano-sized self-assembled structure with hydrophilic domains having a diameter size in the range of 0.5 to 200 nm, due to the presence of a lipophilic additive, and the oil-in-water emulsion contains a thickener or gelling agent in order to create new product consistencies and textures.

### FIELD OF INVENTION

The present invention concerns a viscous or gelified oil-in-water emulsion in which the dispersed oil droplets exhibit a self-assembled internal structure, that allows to create new product consistencies and textures.

### BACKGROUND OF THE INVENTION

### Emulsions in Industry

Emulsions are common colloidal systems in many industrial products such as Food, Cosmetics, Pharmaceutical or Agrochemical preparations. They are often used to deliver functional molecules and nutritional benefits, or to create a certain texture or pleasure to the consumer. Oil-in-water emulsions are made of oil droplets which are dispersed in an aqueous continuous phase and stabilised by surface active molecules. In order to disperse the oil phase into the continuous aqueous phase, homogenisers are used which enable to produce oil droplets in various size ranges (having a radius from ca 100 nm up to several hundreds of micrometers). The surface active material, also denoted as emulsifiers, generally used in oil-in-water based emulsion products can either be low molecular weight hydrophilic surfactants, such as polysorbates, lysolecithins etc, or polymers, such as proteins, e.g. gelatin or from milk, soya, or polysaccharides, hydrocolloids, such as gum arabic or xanthan or particulated materials, such as silica particles, or mixtures thereof.

One relatively new application for emulsions is for the preparation of solid and hydrophilic nano- or microparticles. JP 2004 008837 discloses an oil in water emulsion which contains water-soluble solid particles present in the oil droplets. The particles are in the size range of 20 nm to 10 μm. The particles are prepared in a water-in-oil (w/o) emulsion by means of dehydration (i.e., not a spontaneous process) before the whole particle/oil (S/O) suspension is dispersed in an aqueous phase using the porous membrane emulsification process.

WO 02/076441 discloses the use of an alcohol-in-fluorcarbon microemulsion as a precursor for the preparation of solid nanoparticles The nanoparticles have a diameter below 200-300 nanometres. Nanoparticle formation is not spontaneous and triggered by cooling the precursor microemulsion below about 35° C., or by evaporating the alcohol in the precursor microemulsion or by diluting the microemulsion with a suitable polar solvent.

US 2004/022861 discloses a w/o/w double emulsion, in which the oil droplets containing an aqueous microscopic water phase containing protein or another hydrophilic agent. The whole double emulsion is sprayed into, for instance, liquid nitrogen via a capillary nozzle for production of protein-loaded microparticles.

All these examples describe the non-spontaneous formation of solid hydrophilic (nano)particles using w/o microemulsions or w/o or w/o/w double emulsions and needing an external trigger for the solidification of the hydrophilic domains inside the oil droplets. After preparation of the (nano)particles they are largely unaffected by environmental factors such as temperature, pH, or external fluid properties. It has to be mentioned that ordinary w/o microemulsions in which the water droplets are not solidified, i.e. fluid, are largely affected by such environmental factors.

Oil-in-water emulsion based products are ubiquitous in—Food, Cosmetics, Pharmaceuticals or Agro-chemicals. Prominent oil-in-water emulsion-based food products are for instance milk, mayonnaise, salad dressings, or sauces. Prominent oil-in-water emulsion-based products used in the cosmetical or pharmaceutical Industry are lotions, creams, milks, pills, tablets etc. The oil droplets in such products are usually made of, for instance, triglycerides, diglycerides, waxes, fatty acid esters, fatty acids, essential oils, alcohols, mineral oils, hydrocarbons, or other oily substances.

Emulsions are used either as a starting material, intermediate or final product or as an additive to a final product.

### Emulsion Consistencies

In practice hydrocolloids or polysaccharides are used as thickeners, also denoted as viscosifiers or gelling agents, in order to give oil-in-water emulsions a certain consistency, texture, stability or mouthfeel (Darling D F, Birkett R J; ‘Food colloids in practice’ in ‘Food Emulsions and Foams’ Dickinson E (ed), The Royal Society of Chemistry, London (1987) pp 1-29). Normally, o/w emulsions having a low or moderate oil volume fraction (i.e., amount of oil droplets is below 30-60 vol-%) are quite fluid (liquid) and neither usable as coating material or ointment or cream or gel, nor as product base giving good shelf-life and/or creamy mouthfeel. Such o/w emulsions are often prone to creaming, coalescence, flocculation or sedimentation. In order to create a certain consistency or texture in the emulsion-based product, a thickener is added. The thickener is an ingredient (single component or mixture of components) which does not preferably adsorb to a water-oil interface, i.e., to the interface of the oil droplets, but which essentially provides viscosity to the continuous phase decreasing the Brownian motion of the oil droplets and, in this way, slowing down oil droplet coalescence, sedimentation, flocculation or creaming, and contributing to a better stability and/or creamy sensation.

### DESCRIPTION OF THE INVENTION

The present invention is based on the finding of novel nano-sized self-assembled structures in the interior of oil droplets. The internal droplets structure is formed by the addition of a lipophilic additive (LPA) to the oil droplets. The structures can solubilize lipophilic, amphiphilic and hydrophilic components. The nano-sized self-assembled structures inside the oil droplets mainly consist of nano-sized and thermodynamically stable hydrophilic domains, i.e., water droplets, rods or channels. The nano-sized domains, which are formed spontaneously (thermodynamically driven) inside the emulsion oil droplets, are stabilized by the LPA. The hydrophilic domains can be of the size of 0.5 to 200 nm of diameter, preferably in the range of 0.5 to 150 nm of diameter, even more preferably in the range of 0.5 to 100 nm of diameter, and most preferably in the range of 0.5 to 50 nm.

As used herein, the ‘hydrophilic domain’ consists of the water domains and the hydrophilic headgroup area of the LPA molecules. Due to their ultra-small size, they also exhibit a large surface area which is a suitable location for the solubilization of a variety of different active elements.

The notion ‘self-assembly’ or ‘self-organization’ refers to the spontaneous formation of aggregates (associates) or nano-structures by separate molecules. Molecules in self-assembled structures find their appropriate location based solely on their structural and chemical properties due to given intermolecular forces, such as hydrophobic, hydration or electrostatic forces (Evans, D. F.; Wennerström, H. (Eds.); ‘The Colloidal Domain’, Wiley-VCH, New York, (1999)). The result of self-assembly does not depend on the process of preparation itself and corresponds to a state of minimum energy (stable equilibrium) of the system.

Numerous scientific research has shown that the type of emulsion (o/w or w/o) formed by homogenisation of the respective Winsor system (Winsor I (o/w microemulsion plus excess of oil) or Winsor II (w/o microemulsion plus excess of water)) is the same as that formed in the microemulsion phase which is in equilibrium of its excess continuous phase. For instance, emulsification of a w/o microemulsion plus excess water (Winsor II system) gives at sufficiently high surfactant concentrations, i.e., larger than the critical concentration of the surfactant in the oil phase cμcₒᵢₗ, a w/o emulsion, the continuous phase of which is itself a w/o microemulsion (B. P. Binks, Langmuir (1993) 9, 25-28). This means that when an ordinary w/o microemulsion is diluted with an aqueous phase the formation of a w/o emulsion is preferred over the formation of an o/w emulsion. Binks et al. (B. P. Binks Langmuir (1993) 9, 25-28) explained this behaviour in terms of the partitioning of the surfactant between the water and oil phase in relation to Bancroft's rule (W. D. Bancroft, J. Phys. Chem. (1913) 17, 501): if the surfactant is accumulated in the oil phase, i.e., better soluble in the oil than in the aqueous phase, the formed type of emulsion is always of the w/o and not the o/w-type. In order to form an o/w emulsion from a w/o microemulsion or a Winsor II system (w/o microemulsion plus excess water), it is necessary that the surfactant undergoes a phase inversion, i.e., a change of its solubility from oil-soluble (formation of the w/o emulsion) to water-soluble (formation of a o/w emulsion) (P. Izquierdo et al., Langmuir (2002) 18, 26-30). Using nonionic surfactants such as alkylethoxylates, e.g. the C₁₂EO₄), this can be achieved by cooling the system from 40-50° C. (PIT temperature) down to 25° C. This is completely different from the present invention which correlates the phase behaviour of a lipophilic additive (LPA; forms a w/o microemulsion at room temperature in the oil phase) to the formation of an o/w emulsion in which the oil droplets, containing hydrophilic domains or LPA, are stabilized by an ordinary water-soluble emulsifier. In the present case the hydrophilic domains are fluid and not solid. The w/o microemulsion or the oil containing the hydrophilic domains can be diluted (dispersed) in an aqueous phase without undergoing a phase inversion and loosing the hydrophilic domains inside the dispersed oil droplets, and without the necessity of solidifying the internal hydrophilic domains in the oil droplets before the dispersion step.

According to the invention, the spontaneous formation of the nano-sized self-assembled structure inside the oil droplets of the emulsion of this invention can be realised in different ways. One way is to add a lipophilic additive (LPA) that allows the spontaneous formation of the nano-sized self-assembled structure, to the oil phase prior to the homogenisation step. The other way is to add the lipophilic additive (LPA) to the emulsion product after the homogenisation step. In this case the lipophilic additive will dissolve into the oil droplets and will lead to the spontaneous formation of the nano-sized self-assembled structure inside the oil droplets. As homogeniser, an ordinary industrial or lab-scale homogeniser, such as a Rannie piston homogeniser, a Kinematica rotor stator mixer, a colloid mill, a Stephan mixer, a Couette shear cell or a membrane emulsification device can be taken. Moreover, ultrasound, steam injection or a kitchen mixer are also suitable to produce the emulsion described in this invention. The spontaneous formation of the nano-sized self-assembled structure inside the oil droplets is independent on the energy intake, used to make the emulsion, and the sequence of LPA addition. This means that also Nano and Microfluidics technics are suitable to make the emulsion of this invention.

Heating may also facilitate the dispersion process since the internal structure at high temperature may be less viscous and the dispersion process may require less shear at high temperature.

Another route for making the emulsion of this invention is the use of hydrotropes or water structure breakers, or spontaneous emulsification which can be chemically or thermodynamically driven (Evans, D. F.; Wennerström, H. (Eds.); ‘The Colloidal Domain’, Wiley-VCH, New York, (1999)).

The emulsion systems of this invention are clearly distinguished from emulsions commonly known as water-oil-water double emulsions. w/o/w (water/oil/water) double emulsions are oil-in-water emulsions, in which the oil droplets contain micron-sized water droplets (Garti, N.; Bisperink, C.; Curr. Opinion in Colloid & Interface Science (1998), 3, 657-667). The water droplets inside the dispersed double emulsion oil droplets are prepared (dispersed) by mechanical energy input, e.g., homogenisation, and, as a consequence, are thermodynamically unstable and not self-assembled. The diameter of the inner water droplets in a w/o/w double emulsion is larger than 300 nm diameter. The emulsions of this invention can easily be distinguished from ordinary w/o/w double emulsions since the formation of the nano-sized self-assembled structure inside the oil droplets of the emulsion of this invention is spontaneous and thermodynamically driven, and the mean diameter of the water droplets or channels is below 200 nm.

The present invention is based on the finding that the nano-sized self-assembled structures in the interior of oil droplets is not destroyed when adding common thickeners or gelling agents to the continuous aqueous phase of the emulsion of this invention. Addition of thickeners does not change the self-assembled structures in the interior of the oil droplets. It only gives the fluid emulsions a certain consistency and texture, and as a consequence a better shelf-life, better sensorial properties, the possibility to use the emulsion as coating material (it can easily be sprayed onto a solid surface) or in form of a gel or ointment.

The present invention is concerned with the addition of thickeners, especially sugars, hydrocolloids or polysaccharides or other extended long chain polymers, but also polymers or macromolecules forming a particle gel, such as whey proteins or acidified casein micelles, to the emulsion containing nano-sized self-assembled oil droplets, allows to create new product consistencies and textures. Without a viscosifier the oil-water emulsions at low to medium volume fractions (up to 50% oil volume) are liquid like, i.e. they easily flow under external force fields. Using hydrocolloids in the presence of the oil phase of this invention (oil plus LPA) allows to create gel- or paste-like or highly viscous or viscoelastic materials. Depending on the internal nano-sized self-assembled oil droplet structure, they are acting more as passive (reducing the gel strength) or active (increasing the gel strength) fillers and specifically interact with the gel network. This allows to formulate new products with the advantage of being able to create a high variability of product consistencies while keeping the water and thickener amount in the product constant.

The consistency can, in addition, be tuned by temperature at fixed composition. Depending on the type of added thickener or the type of nano-sized self-assembled structure formed inside the emulsion droplets, increasing temperature reversibly decreases the viscosity of the system, i.e., a paste can become a liquid, or increases the viscosity of the system, i.e., a liquid becomes a gel, or can first decrease the viscosity up to intermediate temperatures before increasing again the viscosity of the system. The viscous or gel properties of the emulsion can easily be modulated by addition of an appropriate thickener and/or forming a certain nano-sized self-assembled structure inside the emulsion droplets.

### Emulsion Formulation

The present invention concerns a viscous or gelified oil-in-water emulsion wherein the oil droplets have a diameter in the range of 5 nm to hundreds of micrometers exhibiting a nano-sized self-assembled structure with hydrophilic domains having a diameter size in the range of 0.5 to 200 nm, due to the presence of a lipophilic additive, and wherein the emulsion contains a thickener in the range of 0.01 and 80 wt-% on the total final product. The thickener concentration is preferably higher than 0.05 wt-%, even more preferably higher than 0.1 wt %, even more preferably higher than 0.5 wt %, and most preferably higher than 1 wt-%. The lower limit depends on the minimal concentration needed to increase the viscosity of the o/w emulsion of this invention. The thickener concentration is preferably lower than 70 wt-%. More preferably the thickener concentration is lower than 60 wt-%. Even most preferably the thickener concentration is lower lower than 50 wt-%. The upper limit depends on the maximal concentration of the thickener which can be added to the o/w emulsion of this invention that still allows producing a homogeneous product. Any combination of the lower and upper limit is comprised in the scope of the present invention.

In certain cases the thickener is added to the formulation. In other cases, the thickener can be already present in the product itself such as a food product, a cream, etc. In the latter case it is not necessary to add it together with the other ingredients of the emulsion of the invention.

Addition of the thickener or gelling agent is to create a certain viscosity in the product. At room temperature, the viscosity is higher than 2 mPas. Preferably the viscosity is higher than 5 mPas. More preferably the viscosity is higher than 10 mPas. Even more preferably the viscosity is higher than 50 mPas, and most preferably higher than 100 mPas. The viscosity can be either the zero shear viscosity, the apparent high shear viscosity, or the complex viscosity. The minimal viscosity values are measured in the system containing only the thickening agent or agents, avoiding the interference of the other ingredients and components (such as oil droplets, emulsifiers etc) on the measured viscosity data.

The LPA can be added as such or made in-situ by chemical, biochemical, enzymatic or biological means. The amount of oil droplets present in the emulsion of this invention (oil droplet volume fraction) is the amount generally used in ordinary oil-in-water emulsion products. The oil in water emulsion of the invention can be either an oil in water emulsion (large oil droplets), a nano oil-in-water emulsion or an oil-in-water microemulsion, depending on the size of the oil droplets.

More precisely, the present invention is directed to oil-in-water emulsions comprising dispersed oil droplets having a nano-sized self-assembled structured interior comprising
(i) an oil selected from the group consisting of mineral oils, hydrocarbons, vegetable oils, waxes, alcohols, fatty acids, mono-, di- or tri-acylglycerols, essential oils, flavouring oils, lipophilic vitamins, esters, neutraceuticals, terpins, terpenes and mixtures thereof,
(ii) a lipophilic additive (LPA) or mixtures of lipophilic and hydrophilic additives, having a resulting HLB value(Hydrophilic-Lipophilic Balance) lower than about 10, preferably lower than 8.
(iii) hydrophilic domains in form of droplets, rods or channels comprising of water or a non-aqueous polar liquid, such as a polyol,
   and
   an aqueous continuous phase which contains an emulsifier and a thickener.

The emulsifier is added to adsorb to the interface of the oil droplets of this invention in order to stabilize them against physical emulsion degradation, e.g, coalsescence and or flocculation. It is selected from the group consisting of low molecular weight surfactants having a HLB>8, proteins from milk, such as whey proteins, whey protein isolates, whey protein concentrates, whey protein aggregates, caseinates, casein micelles, caseins, lysozyme albumins, or from soya, amino acids peptides, protein hydrolysates, block co-polymers, random co-polymers, Gemini surfactants, surface active hydrocolloids such as gum arabic, xanthan gum, gelatin, polyelectrolytes, carrageenans, caboxymethylcellulose, cellulose derivatives, Acacia gum, galactomannans, chitosans, hyaluronc acid, pectins, propylene glycol alginate, modified starches, Portulaca Oleracea, Tragacanth, gellan gum, apoprotein-like biopolymers, such as protein-polysaccharide conjugates or coacervates, or protein-polysaccharide, protein-protein, or polysaccharide-polysaccharide hybrids, conjugates, or mixtures of polymers and biopolymers, polyelectrolyte-surfactant complexes, DNA, nucleic acid, particles (micro or nano-sized), starch and starch-based polymers, amylose, amylopectin and mixtures thereof.

The thickener or gelling agent, is selected from the group consisting of hydrocolloids, polysaccharides, gellan, furcelleran, xanthan gum, carrageenan, carboxymethylcellulose (CMC), micro crystalline cellulose (MCC), galactomannans, guar gum, locust bean gum, hydroxyl propyl methylcellulose (HPMC), starch, maltodextrins, dextrin, dextrose, sugar, invert sugar sirup, sucrose, glucose, glycerol, enzymatically treated starches, starch derivatives, physically modified starch, amylopectin, amylase, agar, tamarind seed gum, konjac gum, gum Arabic, carobseed gum, low and high methoxy pectins, pectin derivatives, propylene glycol alginate (PGA), alginate, gelatine, whey protein particle gels, acid induced casein gels, and mixtures thereof.

As used herein, a ‘lipophilic additive’ (abbreviated also as ‘LPA’) refers to a lipophilic amphiphilic agent which spontaneously forms stable nano-sized self-assembled structures in a dispersed oil phase. The lipophilic additive (mixture) is selected from the group consisting of fatty acids, sorbitan esters, propylene glycol mono- or diesters, pegylated fatty acids, monoglycerides, derivatives of monoglycerides, diglycerides, pegylated vegetable oils, polyoxyethylene sorbitan esters, phospholipids, cephalins, lipids, sugar esters, sugar ethers, sucrose esters, polyglycerol esters and mixtures thereof.

According to the first embodiment of the invention the oil-in-water emulsion exhibits oil droplets having an internal structure taken from the group consisting of the L₂ structure or a combination of a L2 and oil structure (microemulsion or isotropic liquid droplets) in the temperature range of 0° C. to 100° C.

According to the second embodiment of the invention, the oil-in-water emulsion exhibits oil droplets having a L2 structure (microemulsion or isotropic liquid droplets) in the temperature range of 0° C. to 100° C.

According to a third embodiment of the invention, the oil-in-water emulsion exhibits oil droplets having an internal structure taken from the group consisting of the L2 structure (microemulsion or isotropic liquid droplets) or liquid crystalline (LC) structure (e.g. reversed micellar cubic, reversed bicontinuous cubic or reversed hexagonal) and a combination thereof in the temperature range of 0° C. to 100° C.

According to the fourth embodiment of the invention, the oil-in-water emulsion exhibits oil droplets having a LC internal structure in the temperature range of 0° C. to 100° C.

According to a fifth embodiment of the invention, the oil-in-water emulsion exhibits oil droplets having an internal structure taken from the group consisting of the L3 structure, a combination of the L2 and L3 structure, a combination of the lamellar liquid crystalline (Lα) and L2 structure, and a combination of the lamellar crystalline and L2 structure in the temperature range of 0° C. to 100° C.

According to a sixth embodiment of the invention, the oil-in-water emulsion exhibits oil droplets having an internal structure which is a combination of the previously described structures in the temperature range of 0° C. to 100° C.

It is also possible according to the invention, that the oil-in-water emulsion contains further an active element taken from the group consisting of flavors, flavor precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxidants or nutraceuticals selected from the group comprising of lutein, lutein esters, β-carotene, tocopherol, tocopherol acetate, tocotrienol, lycopene, Co-Q₁₀, flax seed oil, lipoic acid, vitamins, polyphenols and their glycosides, ester and/or sulfate conjugates, isoflavones, flavonols, flavanones and their glycosides such as hesperidin, flavan 3-ols comprising catechin monomers and their gallate esters such as epigallocatechin gallate (EGCG),and their procyanidin oligomers, vitamin C, vitamin C palmitate, vitamin A, vitamin B₁₂, vitamin D, α-and γ-polyunsaturated fatty acids, phytosterols, esterified phytosterol, non esterified phytosterol, zeaxanthine, caffeine, and a combination thereof.

All above mentioned internal structures can be without doubt determined by SAXS analysis and by cryo-TEM and freeze fracture EM (Qiu et al. Biomaterials (2000) 21, 223-234, Seddon. Biochimica et Biophysica Acta (1990) 1031, 1-69, Delacroix et al. J. Mol. Biol. (1996) 258, 88-103, Gustafsson et al. Langmuir (1997) 13, 6964-6971, Portes. J. Phys: Condens Matter (1992) 4, 8649-8670) and fast Fourier Transform (FFT) of cryo-TEM images.

For certain applications, the use of temperatures higher than 100° C. (for example retorting temperature or temperature of fusion of crystallinic molecules or temperature of fusion of crystallinic molecules in a media comprising oil or/and LPA) is also possible and is covered by the present invention.

The lipophilic additive (LPA) can also be mixed with a hydrophilic additive (having a HLB larger than 10) up to the amount that the mixture is not exceeding the overall HLB of the mixture of 10 or preferably 8. The additive (mixture) can also be made in-situ by chemical, biochemical, enzymatic or biological means.

The amount of added lipophilic additive is defined as δ. δ is defined as the ratio LPA/(LPA+oil)×100. δ is preferably higher than 0.1, more preferably higher than 0.5, even more preferably higher than 1, even more preferably higher than 3, even more preferably higher than 10 and most preferably higher than 15.

The ratio δ=LPA/(LPA+oil)*100 is preferably lower than 99.9, more preferably lower than 99.5, even more preferably lower than 99.0, even more preferably lower than 95, even more preferably lower than 84, even more preferably lower than 80 and most preferably lower than 70. Any combination of the lower and upper range is comprised in the scope of the present invention. δ can be given either in wt-% or mol-%. The lower and higher limit of δ depends on the properties of the taken oil and LPA, such as the polarity, the molecular weight, dielectric constant, etc., or physical characteristics such as the critical aggregation concentration (cac) or the critical micellar concentration (cmc) of the LPA in the oil droplet phase.

The emulsifier can also be mixed with the LPA, or with the oil, or with the LPA and the oil. This means, that the emulsifier can partly also be present in the interior of the oil droplet and affecting the internal nano-sized self-assembled structure.

The ratio β=emulsifier/(LPA+oil)×100 describes the amount of emulsifier used to stabilize the oil droplets by adsorption with respect to the oil plus LPA content. β is preferably is higher than 0.0001%, preferably higher than 0.001%, preferably higher than 0.01%, preferably higher than 0.1%, preferably higher than 0.5%.

The ratio β=emulsifier/(LPA+oil)×100 preferably lower than 50, more preferably lower than 25 and even more preferably lower than 10%. Any combination of the lower and upper range is comprised in the scope of the present invention. β can be given either in wt-% or mol-%. In certain cases the emulsifier is added to the formulation. In other cases, the emulsifier can be present in the product itself such as a food product, a cream, etc and it is not necessary to add it.

In the oil-in-water emulsion according to the invention, the LPA is selected from the group consisting of myristic acid, oleic acid, lauric acid, stearic acid, palmitic acid, PEG 1-4 stearate, PEG 2-4 oleate, PEG-4 dilaurate, PEG-4 dioleate, PEG-4 distearate, PEG-6 dioleate, PEG-6 distearate, PEG-8-dioleate, PEG-3-16 castor oil, PEG 5-10 hydrogenated castor oil, PEG 6-20 corn oil, PEG 6-20 almond oil, PEG-6 olive oil, PEG-6 peanut oil, PEG-6 palm kernel oil, PEG-6 hydrogenated palm kernel oil, PEG-4 capric/caprylic triglyceride, mono, di, tri, tetraesters of vegetable oil and sorbitol, pentaerythrityl di, tetra stearate, isostearate, oleate, caprylate or caprate, polyglyceryl-3 dioleate, stearate, or isostearate, plyglyceryl 4-10 pentaoleate, polyglyceryl 2-4 oleate, stearate, or isostearate, polyglyceryl 4-10 pentaoleate, polyglycewryl-3 dioleate, polyglyceryl-6 dioleate, polyglyceryl-10 trioleate, polyglyceryl-3 distearate propylene glycol mono- or diesters of C₆ to C₂₀ fatty acid, monoglycerides of C₆ to C₂₀ fatty acid, lactic acid derivatives of monoglycerides, lactic acid derivatives of diglycerides, diacetyl tartaric ester of monoglycerides, triglycerol monostearate cholesterol, phytosterol, PEG 5-20 soya sterol, PEG-6 sorbitan tetra, hexasterarate, PEG-6 sorbitan tetraoleate, sorbitan monolaurate, sorbitan monopalmitate, sorbitan mono trioleate, sorbitan mono and tristearate, sorbitan monoisostearate, sorbitan sesquioleate, sorbitan sesquistearate, PEG-2-5 oleyl ether, POE 2-4 lauryl ether, PEG-2 cetyl ether, PEG-2 stearyl ether, sucrose distearate, sucrose dipalmitate, ethyl oleate, isopropyl myristate, isopropyl palmitate, ethyl linoleate, isopropyl linoleate, poloxamers, phospolipids, lecithins, cephalins, oat lipids and lipophilic amphiphilic lipids from other plants; and mixtures thereof.

The oil-in-water emulsion according to the invention is normally in liquid or semi-liquid form. According to another embodiment of the invention, the emulsion is dried and is available in leaflets, chips or in powder form. Small angle X-ray scattering and Cryo-TEM or freeze fracture EM show that the internal structure of the oil droplets present in the Oil/Water emulsion is reconstituted when it is dried and reconstituted by addition of water.

The oil-in-water emulsion according to the invention is either a final product or an additive. The amount of the additive in the final product is not critical and can be varied.

The emulsion droplets described in this invention can be aggregated or flocculated.

The emulsion described in this invention is a novel type of emulsion which we name ‘ISAMULSION’ to describe the specific nature of the oil droplets containing a structure being Internally Self-Assembled, and to exclude the emulsion of this invention from ordinary oil-in-water or w/o/w double emulsions, including nano- and microemulsions, in which the oil droplets do not have a nano-sized self-assembled structure with hydrophilic domains. The ISAMULSION droplets basically consist of oil droplets which have a nano-sized self-assembled structure with hydrophilic domains. This structure can be of a lamellar liquid crystalline, or a lamellar crystalline, or of a reversed nature comprising the L2, the microemulsion, the isotropic liquid phase, the hexagonal, the micellar cubic, or the bicontinous cubic phase. The structures in the oil phase can appear as a single nano-structure or as a mixture of different nano-structures.

The present invention can be used in Food, Pet Food, Neutraceuticals, Functional Food, Detergents, Nutri-cosmeticals, Cosmetics, Pharmaceuticals, Drug Delivery, Paints, Medical or Agro-chemical Industry, Explosives, Textiles, Mining, Oil well drilling, Paper Industry, Polymer Industry.

The ISAMULSIONS prepared according to the above mentioned examples can be used as such or as an additive.

Having now fully described the invention, it will be understood by those of ordinary skill in the art that the same can be performed within a wide and equivalent range of conditions, formulations and other parameters without affecting the scope of the invention or any embodiment thereof.

### BRIEF DESCRIPTION OF THE DRAWINGS

FIG. 1 shows the structure found in the interior of the ISAMULSION oil droplets as a function of δ=100*LPA/(LPA+oil),

FIG. 2 shows a cryo-TEM image of ISAMULSION oil droplets with no periodic structure (in the presence of a LPA, with nano-structure)(a) in comparison to the corresponding ordinary emulsion droplets (in the absence of a LPA, without nano-structure)(b). Notice that the internal structure that is visible inside the ISAMULSION droplets (FIG. 2a) is invisible in the normal oil droplets (FIG. 2b). The presence of an internal structure can also be visualized by freeze-fracture electron microscopy.

FIG. 3 shows the small angle X-ray scattering (SAXS) pattern of internally structured emulsions.

FIG. 4 shows the structure (measured by SAXS) found in the interior of the emulsified micro-emulsion ISAMULSION oil droplet (L2 phase) in the presence of a κ-carrageenan gel.

FIG. 5 shows the structure (measured by SAXS) found in the interior of ISAMULSION oil droplet having a reversed hexagonal phase (H2) in the presence of a κ-carrageenan gel.

FIG. 6 shows the structure (measured by SAXS) found in ISAMULSION oil droplets (using tetradecane/Dimodan U) having an internal H2 phase, using κ-carrageenan to form the gel.

FIG. 7 shows the structure (measured by SAXS) found in ISAMULSION oil droplets having an internal reversed micellar cubic phase, using κ-carrageenan to form the gel.

FIG. 8 show an emulsified reversed hexagonal phase which is embedded in a MC gel network without being destroyed also upon temperature cycling.

FIG. 9 show an emulsified reversed micellar cubic phase which is embedded in a MC gel network without being destroyed also upon temperature cycling.

FIG. 10 show an emulsified L2 which is embedded in a MC gel network without being destroyed also upon temperature cycling.

FIG. 11 shows an emulsified H2 phase which is embedded in a mixed gel (methylcellulose and κ-carrageenan).

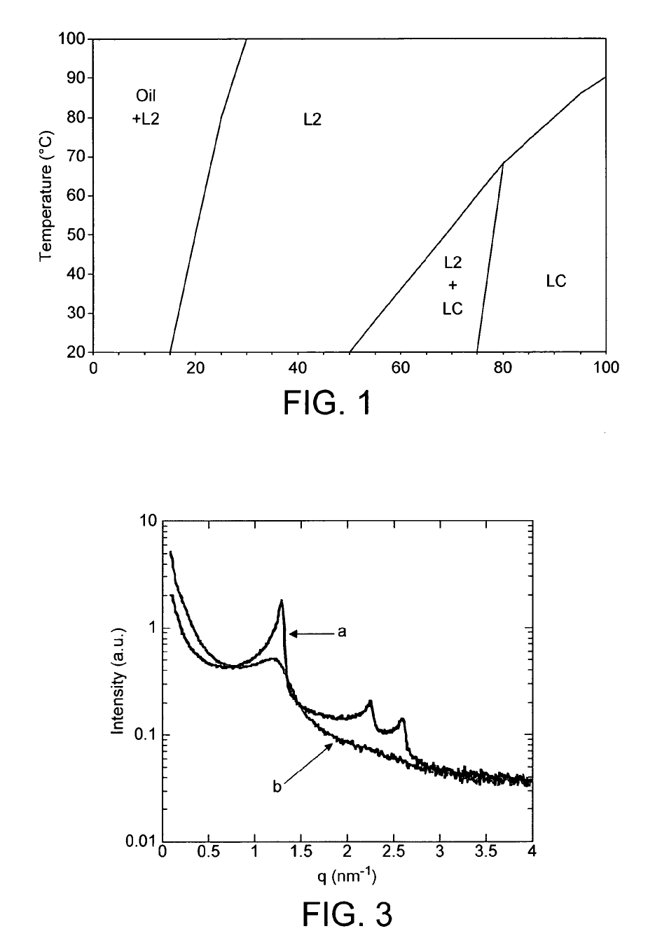

FIG. 1 represents the typical sequence of structures found in the interior of the dispersed oil droplets of the ISAMULSION as a function of the content of the lipophilic additive in % (% LPA =α=100*LPA/(LPA+OIL)) and temperature. L2 denotes a reversed microemulsion-like structure; LC denotes the existence of a liquid crystalline phase or a mixture of different liquid crystalline phases. As FIG. 1 shows, a defined nano-sized self-assembled structure is formed at a given temperature and a specific amount of added lipophilic additive (α value) inside the oil droplets (for a closer description of the mentioned structures, see Evans, D. F.; Wennerström, H. (Eds.); ‘The Colloidal Domain’, Wiley-VCH, New York, (1999)). The amount of added LPA allows to precisely control the type of self-assembly structure, amount of water present in the hydrophilic domains, the amount of internal interface and the size, dimension, of the self-assembly nano-structure formed inside the ISAMULSION droplets. Depending on the oil-type and type of lipophilic additive (LPA), the minimum amount of LPA needed to initiate the spontaneous formation of the self-assembled internal droplet structure is between 0.1 and 5 wt-% on the oil phase.

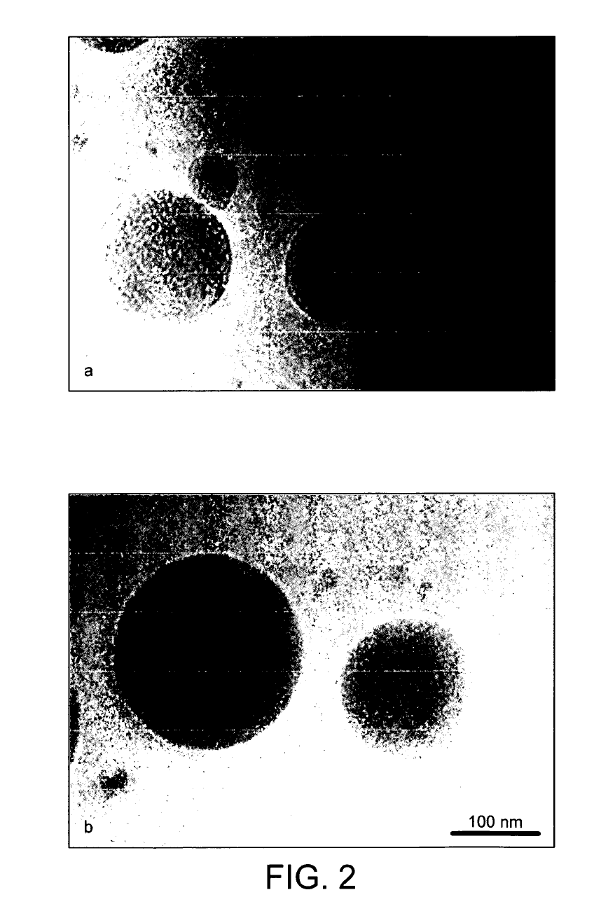

The cryo-TEM image of FIG. 2 was obtained using the standard technique of Adrian et al (Adrian et al. Nature, (1984) 308, 32-36). A home build environmental chamber similar to the one described by Egelhaaf et al (Egelhaaf et al, J. Microsc. (2000) 200, 128-139) was used. The temperature before thinning and vitrifying was set at 25° C. and 100% humidity was used. Frozen grids were stored in liquid nitrogen and transferred into a cryo-holder kept at −180° C. Sample analysis was performed in a Philips CM12 TEM at a voltage of 80 kV. Low dose procedures were applied to minimise beam damage. The ISAMULSION can be identified by the presence of small bright features inside the oil droplets. FIG. 2a is a Cryo-TEM micrographs of an ISAMULSIONs, with no periodic structure, showing characteristic distances between the bright features of about 7-8 nm. It should be noted that such bright features are not observed for standard non-structured emulsions and there is no contrast inside non-structured emulsion droplets (FIG. 2b).

The SAXS curves of FIG. 3 were obtained by a standard equipment (Bergmann et al. J. Appl. Cryst. (2000) 33, 869-875), using a X-ray generator (Philips, P W 1730/10) operating at 40 kV and 50 mA with a sealed-tube Cu anode. A Göbel mirror is used to convert the divergent polychromatic X-ray beam into a focused line-shaped beam of Cu K_{α} radiation (λ=0.154 nm). The 2D scattering pattern is recorded by an imaging-plate detector and integrated to the one-dimensional scattering function I(q) using SAXSQuant software (Anton Paar, Graz, Austria), where q is the length of the scattering vector, defined by q=(4π/λ)sinθ/2, λ being the wavelength and θ the scattering angle. The broad peaks of scattering profiles were desmeared by fitting these data with the Generalized Indirect Fourier Transformation method (Bergmann et al.(2000), 33, 1212-1216). The characteristic distances are given by d=2π/q. FIG. 3 shows the small angle X-ray scattering patterns of ISAMULSIONs.

### EXAMPLES

### Example 1

Generic example using κ-carrageenan to form a gel containing ISAMULSION oil droplets having an internal L2 phase

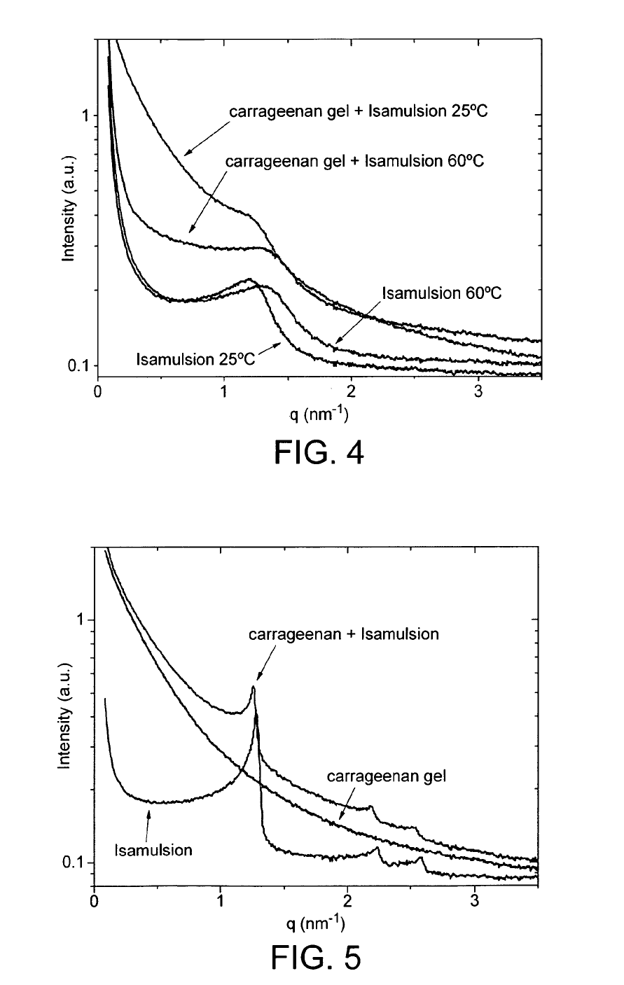

4% of κ-carrageenan was dissolved into pure water under stirring at 50° C. It is a strong gel at ambient temperature. 10 g of ISAMULSIONS were prepared separately by ultrasonication for 20 minutes at 10-%wt dispersed phase. The dispersed phase consists in 0.4625 g R-(+)-Limonene and 0.4625 g Dimodan U (from Danisco) and 0.075 g Pluronic F127 emulsifier (from BASF). The two samples were mixed in the liquid state at 60° C. to form a homogeneous solution. The mixture was then put in the refrigerator to rapidly gelify the system. As shown by SAXS measurements in FIG. 4, an emulsified micro-emulsion can be embedded in the gel network without being destroyed. The internal structure of the ISAMULSION, a L2 structure, is kept as it is in water. The resulting system is a soft gel containing 5% ISAMULSIONS. FIG. 4 shows the structure found in the interior of the ISAMULSION oil droplets in the presence absence of the κ-carrageenan gel.

### Example 2

Generic example of a gel containing ISAMULSION oil droplets having an internal H2 phase, using κ-carrageenan to form the gel

4% of κ-carrageenan was dissolved into pure water under stirring at 50° C. 10 g of ISAMULSIONS were prepared separately by ultrasonication for 20 minutes with 10% wt dispersed phase. The dispersed phase consists in 0.154 g R-(+)-Limonene and 0.771 g Dimodan U and 0.075 g Pluronic F127 emulsifier. The two samples were mixed in the liquid state at 60° C. to form a homogeneous solution. The mixture was put in the fridge for rapidly gelify the polymer. As shown in FIG. 5 by SAXS measurement, an emulsified reversed hexagonal phase can be embedded in the gel network without being destroyed. The internal structure of the ISAMULSION is kept as it is in water. The resulting system is a soft gel containing 5% ISAMULSIONS. FIG. 5 shows the structure found in the interior of the ISAMULSION oil droplets, in the κ-carrageenan gel alone at 25° C. and in the the mixed ISAMULSION gel system.

### Example 3

Generic example of a gel containing ISAMULSION oil droplets (consisting of tetradecane/Dimodan U) having an internal H2 phase, using κ-carrageenan to form the gel.

4% of κ-carrageenan was dissolved into pure water under stirring at 50° C. 10 g of ISAMULSIONS were prepared separately by ultrasonication for 20 minutes at 10% wt dispersed phase. The dispersed phase consists of 0.139 g Tetradecane, i.e., another oil than used in example 2, and 0.786 g Dimodan U and 0.075 g Pluronic F127 emulsifier. The two samples were mixed in the liquid state at 60° C. to form a homogeneous solution. The mixture was put in the fridge for rapidly gelify the system.

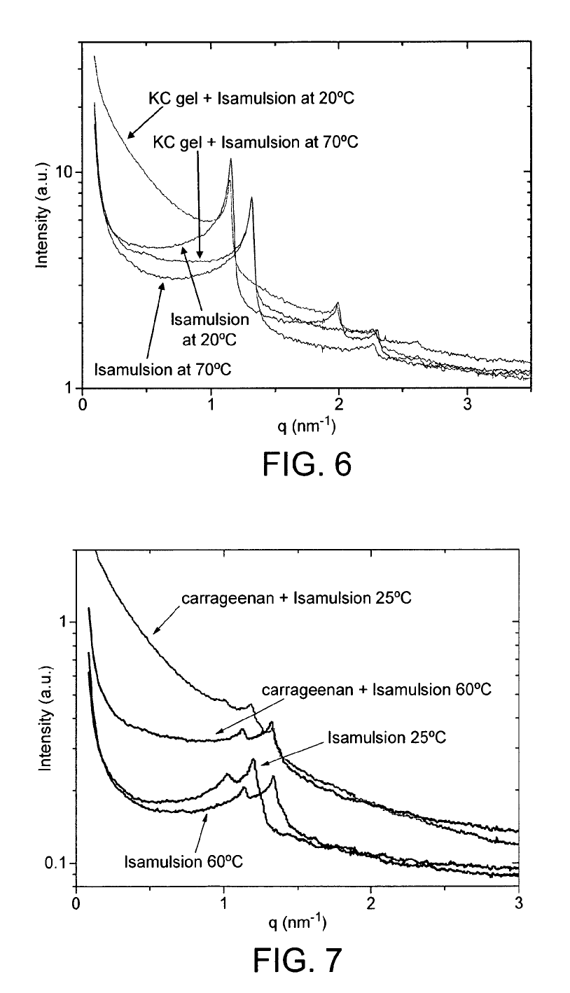

As shown by SAXS measurement in FIG. 6, an emulsified hexagonal phase can be embedded in the gel network without being destroyed. The internal structure of the ISAMULSION is kept as it is in water. The resulting system is a soft gel containing 5% ISAMULSIONS. FIG. 6 shows the structure found in the interior of the ISAMULSION oil droplets, the signal of the κ-carrageenan gel alone at 25° C., and the signal of the mixed ISAMULSION gel system.

### Example 4

Generic example of a gel containing ISAMULSION oil droplets having an internal micellar cubic phase, using κ-carrageenan to form the gel.

4% of κ-carrageenan was dissolved into pure water under stirring at 50° C. 10 g of ISAMULSIONS were prepared separately by ultrasonication for 20 minutes at 10% wt dispersed phase. The dispersed phase consists in 0.264 g R-(+)-Limonene and 0.661 g Dimodan U and 0.075 g Pluronic F127 emulsifier. The two samples were mixed in the liquid state at 60° C. to form a homogeneous solution. The mixture was put in the fridge for rapid gelification of the polymer. As shown by SAXS measurement in FIG. 7, an emulsified micellar cubic phase can be embedded in the gel network without being destroyed. The internal structure of the ISAMULSION is kept as it is in water. The resulting system is a soft gel containing 5% ISAMULSIONS. FIG. 7 shows the structure found in the interior of the ISAMULSION oil droplets in the absence and presence of the gel at 25° C. and 60° C.

The size of these ISAMULSION droplets were measured by dynamic light scattering to be 76.2 nm. When increasing temperature in the mixed gel, the system gets more liquid. This liquid was diluted 4000 times. Dynamic light scattering of this diluted solution gives exactly the same droplet size as measured in the ISAMULSIONS in the absence of the thickener or gelling agent, namely 74.8 nm. Thus, the ISAMULSION droplet size is kept the same also after gelification of the system.

### Example 5

Generic example using methylcellulose (MC) as gelifying polymer.

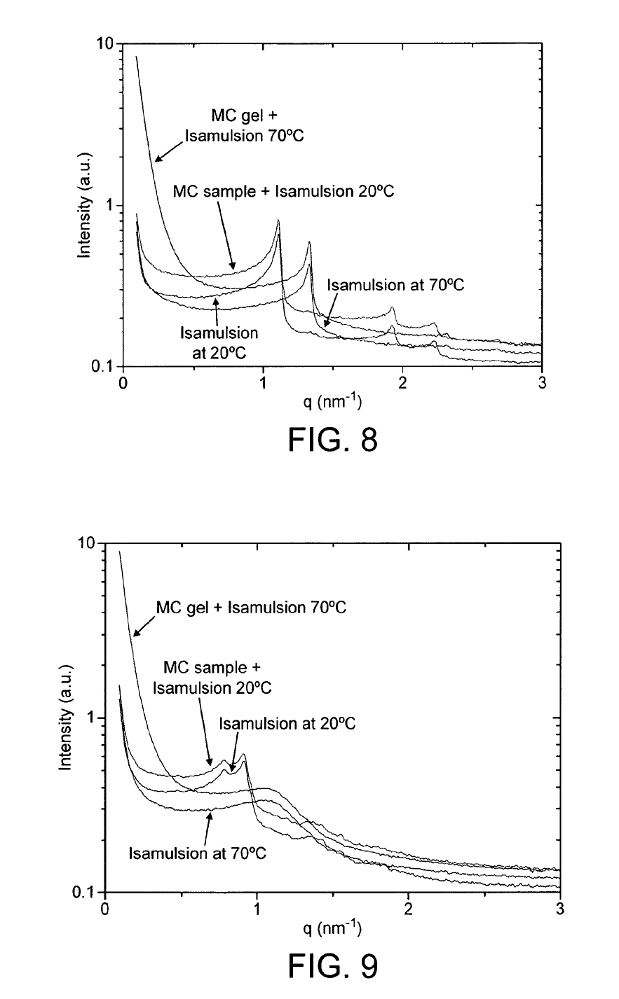

4% of methylcellulose was dissolved in pure water with vigorous stirring at 60° C. and was left to cool down to room temperature while continuously stirring the system. 10 g of a ISAMULSION sample containing 10 wt % dispersed phase was prepared separately by ultrasonication for 20 minutes. The ISAMULSION sample contained 0.139 g tetradecane, 0.786 g Dimodan U and 0.075 g Pluronic F127 emulsifier giving a reversed hexagonal internal droplet phase. The two samples were mixed in the liquid state at 20° C. to form a homogeneous solution. The mixture was heated to 70° C. to gelify the polymer. As shown by SAXS measurements in FIG. 8 an emulsified hexagonal phase can be embedded in the MC gel network without being destroyed. The internal structure of the ISAMULSION is kept as it is in water. The resulting system is a soft gel containing 5% ISAMULSION. FIG. 8 shows the structure found in the interior of the ISAMULSION oil droplets and in the interior of the ISAMULSION oil droplets mixed into the gel.

The size of these ISAMULSION oil droplets was found to be 153 nm (measured by dynamic light scattering). When the latter sample was mixed with the MC sample the obtained size was 148 nm. After gelifying and degelifying, i.e., increasing and subsequently decreasing of the temperature the droplet size was 147 nm, i.e. the same as before the temperature cycling. All the samples were diluted 4000 times before the dynamic light scattering experiment was performed. The scattering particle size polydispersity was around 30% in all cases. These results confirm that the MC/MC gel does not influence the internal structure of the ISAMULSIONS oil droplets when embedding them into the gel.

### Example 6

Generic example using methylcellulose (MC) as thickener.

4% of methylcellulose was dissolved in pure water by vigorous stirring at 60° C. Then the system was left to cool down to the room temperature while continuing stirring. 10 g of an ISAMULSION sample having 10 wt % of a dispersed phase was prepared separately by ultrasonication for 20 minutes. The ISAMULSUION contained 0.277 g tetradecane, 0.648 g Dimodan U and 0.075 g Pluronic F127 emulsifier. The two samples were mixed in the liquid state at 20° C. to form a homogeneous solution. The mixture was heated to 70° C. for rapidly gelifying the polymer. As shown by SAXS measurements in FIG. 9 an emulsified micellar cubic phase can be embedded in the MC gel network without being destroyed. The internal structure of the ISAMULSION is kept as it is in water and also the same transition from emulsified micellar cubic phase to emulsified micro-emulsion is observed in both cases. The resulting system is a soft gel containing 5% ISAMULSIONS. The size of these ISAMULSIONS was found by dynamic light scattering to be 156 nm. When this sample was mixed with the MC sample the obtained size was 159 nm. Both samples were diluted 4000 times before the dynamic light scattering experiment. The obtained size polydispersity was around 30% in both cases. These results confirm that the MC/MC gel does not influence the ISAMULSION internal droplet structure.

### Example 7

Generic example using methylcellulose (MC) as gelling agent.

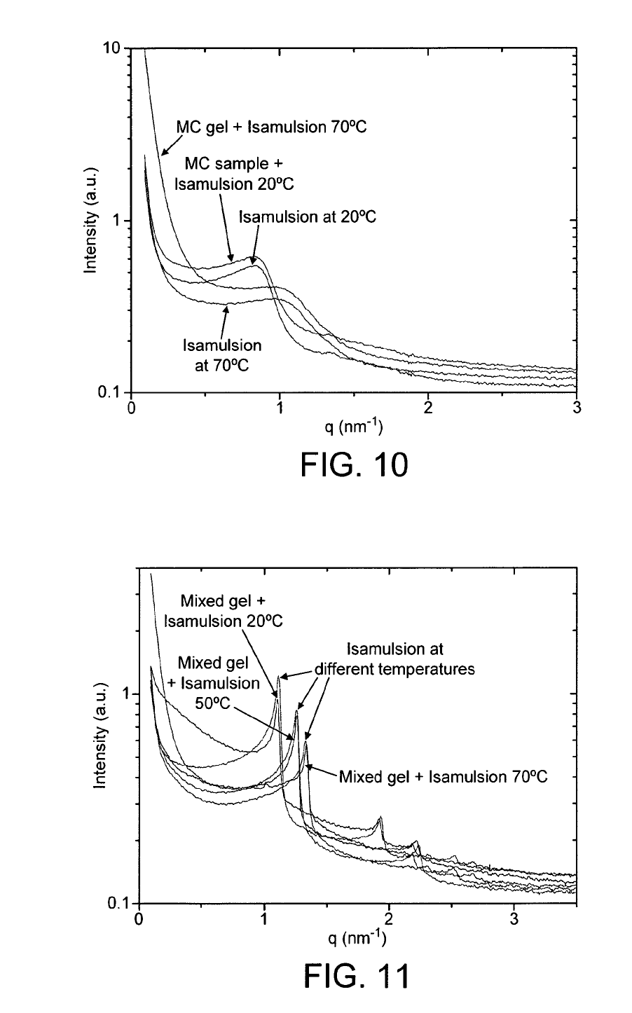

4% of methylcellulose was dissolved in pure water while vigorous stirring the system at 60° C. C. Then the system was left to cool down to the room temperature while continuing stirring. 10 g of an ISAMULSION having 10% wt of a dispersed phase were prepared separately by ultrasonication for 20 minutes. The ISAMULSION contained 0.416 g tetradecane, 0.509 g Dimodan U and 0.075 g Pluronic F127 emulsifier. The two samples were mixed in the liquid state at 20° C. to form a homogeneous solution. The mixture was heated to 70° C. to gelify the polymer. As shown by SAXS measurement in FIG. 10 an emulsified micro-emulsion (L2) can be embedded in the gel network without being destroyed. The internal structure of the ISAMULSION is kept as it is in water. The resulting system is a soft gel containing 5% ISAMULSION.

The size of these ISAMULSIONS was found by dynamic light scattering to be 148 nm. When this sample was mixed with the MC sample the obtained size was also 148 nm. Both samples were diluted 4000 times before the dynamic light scattering experiment. The obtained size polydispersity was around 30% in both cases. These results confirm that the MC/MC gel does not influence the ISAMULSION internal droplet structure.

### Example 8

Generic example using a mixture of methylcellulose (MC) and κ-carrageenan (KC) as gelifying polymers.

2% of MC and 2% of KC was dissolved in pure water under stirring at 60° C. C. Then the system was left to cool down to the room temperature while continuing stirring. 10 g of an ISAMULSION containing 10 wt % dispersed phase were prepared separately by ultrasonication for 20 minutes. The ISAMULSION contained 0.139 g tetradecane and 0.786 g Dimodan U and 0.075 g Pluronic F127 emulsifier. The two samples were mixed at 50° C. to form a homogeneous sample. The obtained mixture containing 1% of MC and 1% of KC is liquid in a narrow temperature regime around 50° C. The sample was measured at 20° C. (KC driven gel), 50° C. (liquid) and at 70° C. (MC driven gel). As shown by SAXS measurement in FIG. 11 an emulsified reversed hexagonal phase can be embedded in the mixed gel network without being destroyed. The internal structure of the ISAMULSION oil droplets is kept as it is in water.

The size of the ISAMULSION droplets was found by dynamic light scattering to be 148 nm. When this sample was mixed with the MC sample the obtained size was also 148 nm. Both samples were diluted 4000 times before performing the dynamic light scattering experiment and showed a size polydispersity of around 30%. These results confirm that the mixed MC-KC gel does not influence the ISAMULSION internal oil droplet structure.

1. Oil-in-water emulsion comprising oil droplets having a diameter in the range of 5 nm to hundreds of micrometers exhibiting a nano-sized self-assembled structure with hydrophilic domains having a diameter size in the range of 0.5 to 200 nm, due to the presence of a lipophilic additive, and comprising a component selected from the group consisting of a thickener and gelling agent comprising 0.01 and 80 wt-% of the total final product.

2. Oil-in-water emulsion according to claim 1, wherein the thickener is selected from the group consisting of hydrocolloids, polysaccharides, gellan, furcelleran, xanthan gum, carrageenan, carboxymethylcellulose (CMC),micro crystalline cellulose (MCC), galactomannans, guar gum, locust bean gum, hydroxyl propyl methylcellulose (HPMC), starch, maltodextrins, dextrin, dextrose, sugar, invert sugar, sugar syrup, sucrose, glucose, glycerol, enzymatically treated starches, starch derivatives, physically modified starch, amylopectin, amylase, agar, tamarind seed gum, konjac gum, gum Arabic, carobseed gum, low and high methoxy pectins, pectin derivatives, propylene glycol alginate (PGA), alginate, gelatine, whey protein particle gels, acid induced casein gels, and mixtures thereof.

3. Oil-in-water emulsion according to claim 1, comprising dispersed oil droplets having a nano-sized self-assembled structured interior comprising
(i) an oil selected from the group of consisting of mineral oils, hydrocarbons, vegetable oils, waxes, alcohols, fatty acids, mono-, di-, tri-acylglycerols, essential oils, flavouring oils, lipophilic vitamins, esters, neutraceuticals, terpins, terpenes and mixtures thereof.
(ii) a lipophilic additive (LPA) or mixtures of lipophilic and hydrophilic additives, having a resulting HLB value (Hydrophilic-Lipophilic Balance) lower than about 10,
(iii) hydrophilic domains or a non-aqueous polar liquid.
and
an aqueous continuous phase comprising an emulsifier.

4. Oil-in-water emulsion according to claim 1, wherein the emulsifier is selected from the group consisting of low molecular weight surfactants having a HLB>8, proteins from milk, protein hydrolysates, block co-polymer, random co-polymers, Gemini surfactants, surface active hydrocolloids apoprotein-like biopolymers, polyelectrolytesurfactant complexes, DNA, nucleic acid, particles (micro or nano-sized), starch and starch-based polymers, amylose, amylopectin and mixtures thereof.

5. Oil-in-water emulsion according to claim 1 4, wherein the oil droplets have an internal structure selected from the group consisting of L2 structure, a combination of L2 and oil structure in the temperature range of 0° C. to 100° C.

6. Oil-in-water emulsion according to claim 1 4, wherein the oil droplets have an L2 internal structure in the temperature range of 0° C. to 100° C.

7. Oil-in-water emulsion according to claim 1, wherein the oil droplets have an internal structure selected from the group consisting of L2 structure, LC structure and a combination thereof in the temperature range of 0° C. to 100° C.

8. Oil-in-water emulsion according to claim 1, wherein the oil droplets have a LC internal structure in the temperature range of 0° C. to 100° C.

9. Oil-in-water emulsion according to claim 1, wherein the oil droplets have an internal structure selected from the group consisting of L3 structure, a combination of L2 and L3 structure, a combination of Lα and L2 structure, and a combination of lamellar crystalline structure and L2 structure in the temperature range of 0° C. to 100° C.

10. Oil-in-water emulsion according to claim 1, wherein the emulsion contains an active element selected from the group consisting of flavors, flavor precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxydants or nutraceuticals, isoflavones, flavonols, flavanones and their glycosides, flavan 3-ols, vitamin C, vitamin C palmitate, vitamin A, vitamin B₁₂, vitamin D, α-and γ-polyunsaturated fatty acids, phytosterols, esterified phytosterol, non esterified phytosterol, zeaxanthine, caffeine, and mixtures thereof.

11. Oil-in-water emulsion according to claim 1, wherein the LPA is selected from the group consisting of long-chain alcohols, fatty acids, pegylated fatty acids, glycerol fatty acid esters, monoglycerides, diglycerides, derivatives of mono-diglycerides, pegylated vegetable oils, sorbitan esters, polyoxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, and polyglycerol esters.

12. Oil-in-water emulsion according to claim 11, wherein the LPA is selected from the group consisting of myristic acid, oleic acid, lauric acid, stearic acid, palmitic acid, PEG 1-4 stearate, PEG 2-4 oleate, PEG-4 dilaurate, PEG-4 dioleate, PEG-4 distearate, PEG-6 dioleate, PEG-6 distearate, PEG-8-dioleate, PEG-3-16 castor oil, PEG 5-10 hydrogenated castor oil, PEG 6-20 corn oil, PEG 6-20 almond oil, PEG-6 olive oil, PEG-6 peanut oil, PEG-6 palm kernel oil, PEG-6 hydrogenated palm kernel oil, PEG-4 capric/caprylic triglyceride, mono, di, tri, tetraesters of vegetable oil and sorbitol, pentaerythrityl di, tetra stearate, isostearate, oleate, caprylate or caprate, polyglyceryl-3 dioleate, stearate, or isostearate, plyglyceryl 4-10 pentaoleate, polyglyceryl 2-4 oleate, stearate, or isostearate, polyglyceryl 4-10 pentaoleate, polyglycewryl-3 dioleate, polyglyceryl-6 dioleate, polyglyceryl-10 trioleate, polyglyceryl-3 distearate propylene glycol mono- or diesters of C₆ to C₂₀ fatty acid, monoglycerides of C₆ to C₂₀ fatty acid, lactic acid derivatives of monoglycerides, lactic acid derivatives of diglycerides, diacetyl tartaric ester of monoglycerides, triglycerol monostearate cholesterol, phytosterol, PEG 5-20 soya sterol, PEG-6 sorbitan tetra, hexasterarate, PEG-6 sorbitan tetraoleate, sorbitan monolaurate, sorbitan monopalmitate, sorbitan mono trioleate, sorbitan mono and tristearate, sorbitan monoisostearate, sorbitan sesquioleate, sorbitan sesquistearate, PEG-2-5 oleyl ether, POE 2-4 lauryl ether, PEG-2 cetyl ether, PEG-2 stearyl ether, sucrose distearate, sucrose dipalmitate, ethyl oleate, isopropyl myristate, isopropyl palmitate, ethyl linoleate, isopropyl linoleate, poloxamers, phospolipids, lecithins, cephalins, oat lipids and lipophilic amphiphilic lipids from other plants, and mixtures thereof.

13. Oil-in-water emulsion according to claim 1, wherein the emulsion comprises a hydrophilic emulsifier selected from the group consisting of low molecular weight surfactants having a HLB>8, proteins from milk or soya, peptides, protein hydrolysates, block co-polymers, surface active hydrocolloids, nano or micro-particles and mixtures thereof.

14. Powder comprising oil droplets having a diameter in the range of 5 nm to hundreds of micrometers exhibiting a nano-sized self-assembled structure with hydrophilic domains having a diameter size in the range of 0.5 to 200 nm, due to the presence of a lipophilic additive, and comprising a component selected from the group consisting of a thickener and gelling agent comprising 0.01 and 80 wt-% of the total final product that has been dried and is in a powder form.

15. Oil-in-water emulsion according to claim 1, wherein it is a final product.

16. Oil-in-water emulsion according to claim 1, wherein it is in a form selected from the group consisting of a starting material, an intermediate product and an additive to a final product.

17. Oil-in-water emulsion according to claim 4, wherein the emulsifier is selected from the group consisting of whey proteins, whey protein isolates, whey protein concentrates, whey protein aggregates, caseinates, casein micelles, caseins, lysozyme, albumins, proteins from soya, amino acids peptides, gum arabic, xanthan gum, gelatin, polyelectrolytes, carrageenans, caboxymethylcellulose, cellulose derivatives, Acacia gum, galactomannans, chitosans, hyaluronc acid, pectins, propylene glycol alginate, modified starches, Portulaca Oleracea, Tragacanth, gellan gum, protein-polysaccharide conjugates or coacervates, protein-polysaccharide, protein-protein, polysaccharide-polysaccharide hybrids, conjugates, mixtures of polymers and biopolymers, and mixtures thereof.

18. Oil-in-water emulsion according to claim 10, wherein the active element is selected from the group consisting of lutein, lutein esters, β-carotene, tocopherol, tocopherol acetate, tocotrienol, lycopene, Co-Q₁₀, flax seed oil, lipoic acid, vitamins, polyphenols and their glycosides, ester and/or sulfate conjugates, hesperidin, catechin monomers and their gallate esters, epigallocatechin gallate and their procyanidin oligomers, and combinations thereof.
